(19) United States
(12) Patent Application Publication
(10) Pub. No.: US 2013/0178741 A1
(43) Pub. Date: Jul. 11, 2013

(54) METHOD AND APPARATUS FOR ULTRASOUND VOLUME IMAGE DATA PROCESSING

(71) Applicant: GE MEDICAL SYSTEMS GLOBAL TECHNOLOGY COMPANY, LLC, Waukesha, WI (US)

(72) Inventors: Gang LIU, Shen Yang (CN); Yiming ZHAO, Zhou Shan (CN); Houbing LIU, Wu Xi (CN); Shuxiu WANG, Wu Xi (CN)

(73) Assignee: GE MEDICAL SYSTEMS GLOBAL TECHNOLOGY COMPANY, LLC, Waukesha, WI (US)

(21) Appl. No.: 13/712,025

(22) Filed: Dec. 12, 2012

(30) Foreign Application Priority Data
Dec. 12, 2011 (CN) 201110462297.X

(51) Int. Cl. A61B 8/08

(52) U.S. Cl. CPC A61B 8/483; A61B 8/5207
USPC 600/443

(57) ABSTRACT

A method for ultrasound volume image data processing comprising acquiring ultrasound volume image data in a scan volume in which a line-like object is located, locating a position of the line-like object based on the ultrasound volume image data, defining a slab boundary box surrounding the line-like object based on the position of the line-like object, and rendering a region surrounded by the slab boundary box.

### BACKGROUND OF THE INVENTION

Embodiments of the present invention generally relate to a method and a system for ultrasound volume image data processing and, in particular, to a method and an apparatus for ultrasound volume image data processing in a volume ultrasound scan mode.

Currently, the display mode of a blood vessel based on ultrasound scan includes a 2D slice mode and a 3D rendering mode. For a 2D slice mode, as a blood vessel is often curvous and has branches, it is hard to display the whole vessel while the blood vessel is thin.

In a 3D rendering mode, minimum intensity projection is used to display blood vessels, mainly focusing on liver blood vessels because tissues are uniform and have high contrast in a liver. But for leg and arm blood vessels, the minimum intensity projection mode has poor image quality because the dark tissues around a blood vessel have a big effect on the blood vessel.

### BRIEF SUMMARY OF THE INVENTION

According to an embodiment of the present invention, there is provided a method for ultrasound volume image data processing. The method comprises acquiring ultrasound volume image data in a scan volume in which a line-like object is located, locating a position of the line-like object based on the ultrasound volume image data, defining a slab boundary box surrounding the line-like object based on the position of the line-like object, and rendering a region surrounded by the slab boundary box.

According to an embodiment of the present invention, there is provided an ultrasound volume image data processing device. The ultrasound volume image data processing device comprises an ultrasound volume image data acquisition unit configured to acquire ultrasound volume image data in a scan volume in which a line-like object is located, an object locating unit configured to locate a position of the line-like object based on the ultrasound volume image data, a slab boundary box determination unit configured to define a slab boundary box surrounding the line-like object based on the position of the line-like object, and a rendering unit configured to render a region surrounded by the slab boundary box.

### BRIEF DESCRIPTION OF THE DRAWINGS

In the drawings:

FIG. 1 illustrates a method for the ultrasound volume image data processing in the volume ultrasound scan mode according to an embodiment of the present invention;

FIG. 2 illustrates CF mode scan planes according to an embodiment of the present invention, where there are two CF mode scan planes (left and right) at the volume ultrasound scan boundary;

FIG. 3 illustrates a method for reducing the tissue image data in the CF mode image data according to an embodiment of the present invention;

FIGS. 4A and 4B illustrate respectively a filter used in the embodiment of FIG. 3 according to an embodiment of the present invention;

FIG. 5 illustrates a method for determining the type of the blood vessel according to an embodiment of the present invention;

FIG. 6 illustrates a method for determining the position of the blood vessel according to an embodiment of the present invention;

FIG. 7 illustrates comparison between the ultrasound image based on Hessian matrix processing and the ultrasound image before the Hessian matrix processing according to an embodiment of the present invention;

FIG. 8 illustrates a method for defining a slab boundary box according to an embodiment of the present invention;

FIG. 9 illustrates conversion from an acquisition coordinate system to a Cartesian coordinate system according to an embodiment of the present invention;

FIG. 10 illustrates a schematic diagram of determination of the plane vector of a pair of outer surfaces of the slab boundary box based on the direction of the center point connecting line of the blood vessel according to an embodiment of the present invention;

FIGS. 11A and 11B illustrate respectively the minimum intensity projection images of a side view and a top view according to an embodiment of the present invention; and

FIG. 12 illustrates a blood vessel image generation system according to an embodiment of the present invention.

### DETAILED DESCRIPTION OF THE INVENTION

The present invention will be described below by way of some embodiments, wherein details thereof are used to facilitate understanding the present invention rather than limit the present invention. Some embodiments of the present invention provide a method and an apparatus for ultrasound volume image data processing in a volume ultrasound scan mode.

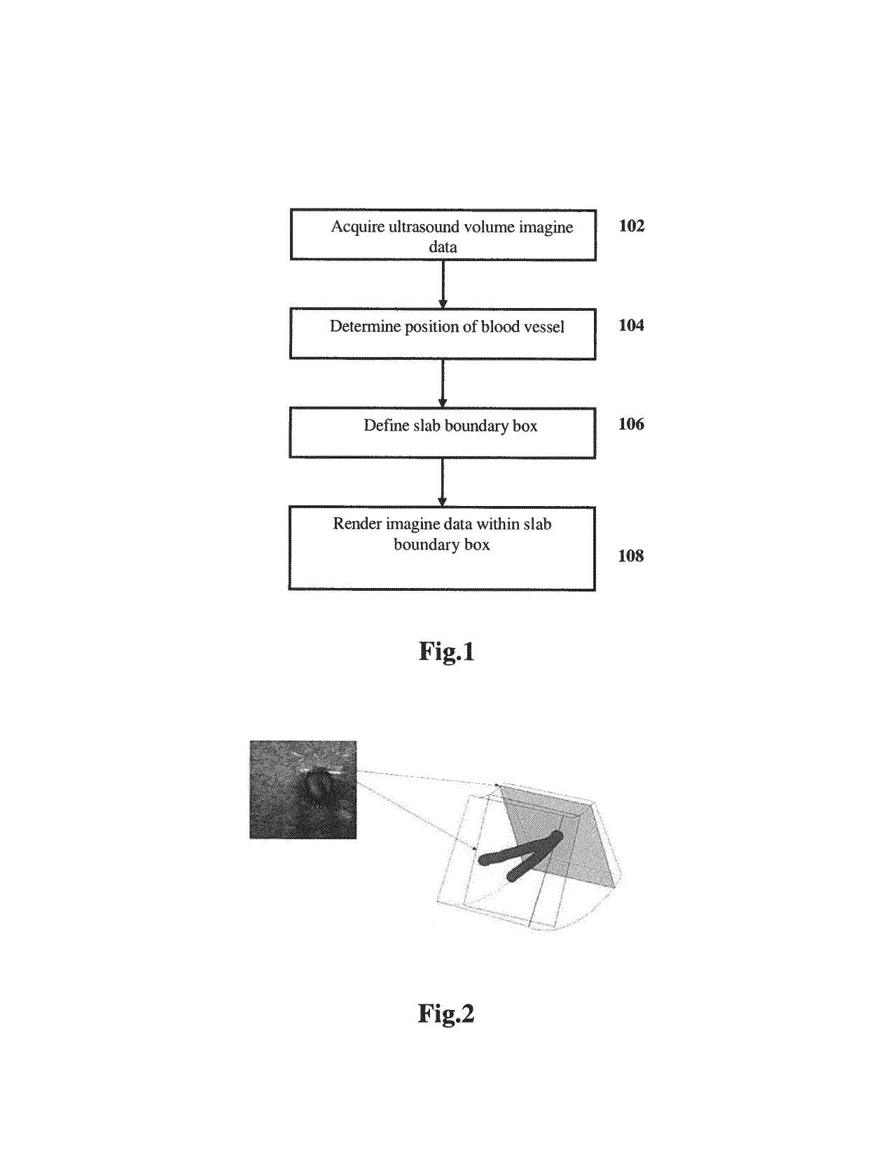

FIG. 1 illustrates a method for the ultrasound volume image data processing in the volume ultrasound scan mode according to an embodiment of the present invention. At Step 102, the ultrasound volume image data in the volume ultrasound scan mode is acquired. At Step 104, the position of the blood vessel is determined based on the acquired ultrasound volume image data in the volume ultrasound scan mode. At Step 106, the slab boundary box surrounding the blood vessel is determined based on the position of the blood vessel. At Step 108, the region in the range of the slab boundary box is rendered.

According to an embodiment of the present invention, acquisition of the ultrasound volume image data in the volume ultrasound scan mode includes setting up a specific scan mode like a B mode, a CF mode to acquire the ultrasound volume image data in the volume ultrasound scan mode. In an embodiment, a volume ultrasound scan probe sweeps in the direction parallel to the long axis azimuth of the blood vessel.

By sampling the object to be imaged in the B mode, the image data is generated based on the magnitude of the ultrasound echo, and the B mode ultrasound volume image data in the B mode is obtained. The obtained B mode ultrasound volume image data may be saved in the memory for further processing.

By sampling the object to be imaged in the CF mode, the image data is generated based on the phase of the Doppler component of the ultrasound echo, and the CF mode image data in the CF mode is obtained. The obtained CF mode image data may be saved in the memory for further processing. In an embodiment, two CF mode scan planes are set up at the boundary of the sweeping scan volume range in the volume ultrasound scan mode to acquire the image data of two CF mode planes in the CF mode, and the obtained image data of two CF mode planes is saved for further processing. FIG. 2 illustrates CF mode scan planes according to an embodiment of the present invention, where there are two CF mode scan planes (left and right) at the volume ultrasound scan boundary.

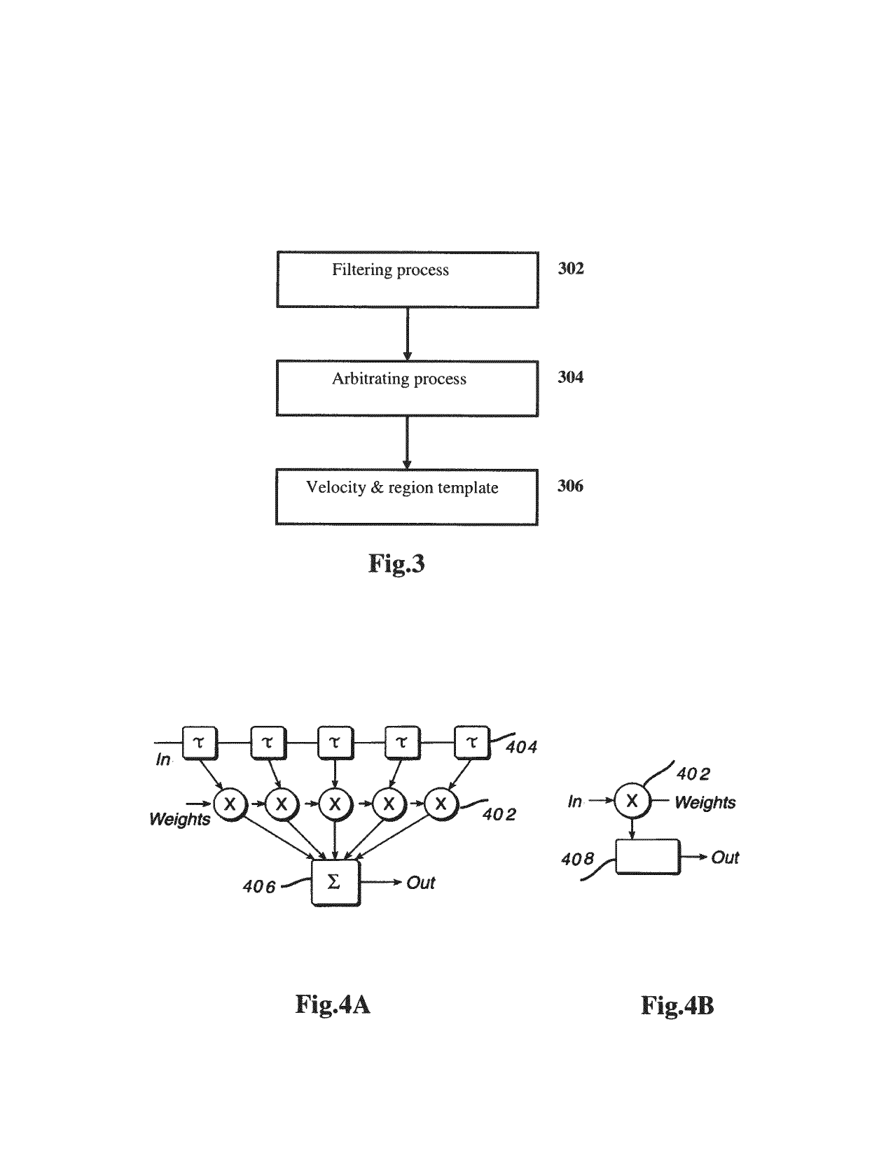

FIG. 3 illustrates a method for reducing the tissue image data in the CF mode image data according to an embodiment of the present invention. At Step 302, predetermined filter characteristic parameters are set up for filtering the CF mode image data. In an embodiment, the CF mode image data is wall filtered. The wall filter is a highpass filter with an adjustable cutoff frequency. Since the tissue image data has higher frequency characteristic than the blood flow image data in the blood vessel, the tissue image data will be effectively suppressed by setting up a predetermined cutoff frequency. At Step 304, power arbitration is performed on the CF mode image data filtered as described above. In an embodiment, the predetermined threshold is set up, and it is determined whether the filtered CF mode image data is larger than the predetermined threshold. If it is larger than the predetermined threshold, it is considered as corresponding to the valid blood flow image data of the blood vessel; otherwise it is considered as not corresponding to the invalid blood flow image data of the blood vessel. At Step 306, a velocity and region template is obtained based on the power arbitration result, in which the valid blood flow corresponding to the blood vessel is marked with “1” and the invalid blood flow not corresponding to the blood flow is marked with “0”. The invalid blood flow image data not corresponding to the blood vessel is removed, thereby obtaining the remaining valid blood flow image data corresponding to the blood vessel.

FIGS. 4A and 4B illustrate respectively a filter used in the embodiment of FIG. 3 according to an embodiment of the present invention. The embodiment of FIG. 4A illustrates a Finite Pulse Response (FIR) filter. The CF image data in the volume ultrasound scan mode obtained at Step 102 of FIG. 1 is input (In) in the delay stage chain 404 and is multiplied by a weight factor in a multiplier 402 when passing through the delay stage to provide a desired filter characteristic for the CF image data. Then the obtained product is summed in the summing stage 406 to obtain the filtered output (Out) data. The embodiment of FIG. 4B illustrates a filter including a multiplier 402 and an accumulator 408. In this embodiment, the CF image data in the volume ultrasound scan mode obtained at Step 102 of FIG. 1 are multiplied by the weight factor applied to the multiplier in order, and their products are accumulated in the accumulator 408. The multiplier-accumulator embodiment may be cascaded. According to an embodiment of the present invention, the blood vessel image data and the tissue image data in the CF image data may be appropriately separated by selecting a proper delay coefficient or weight factor.

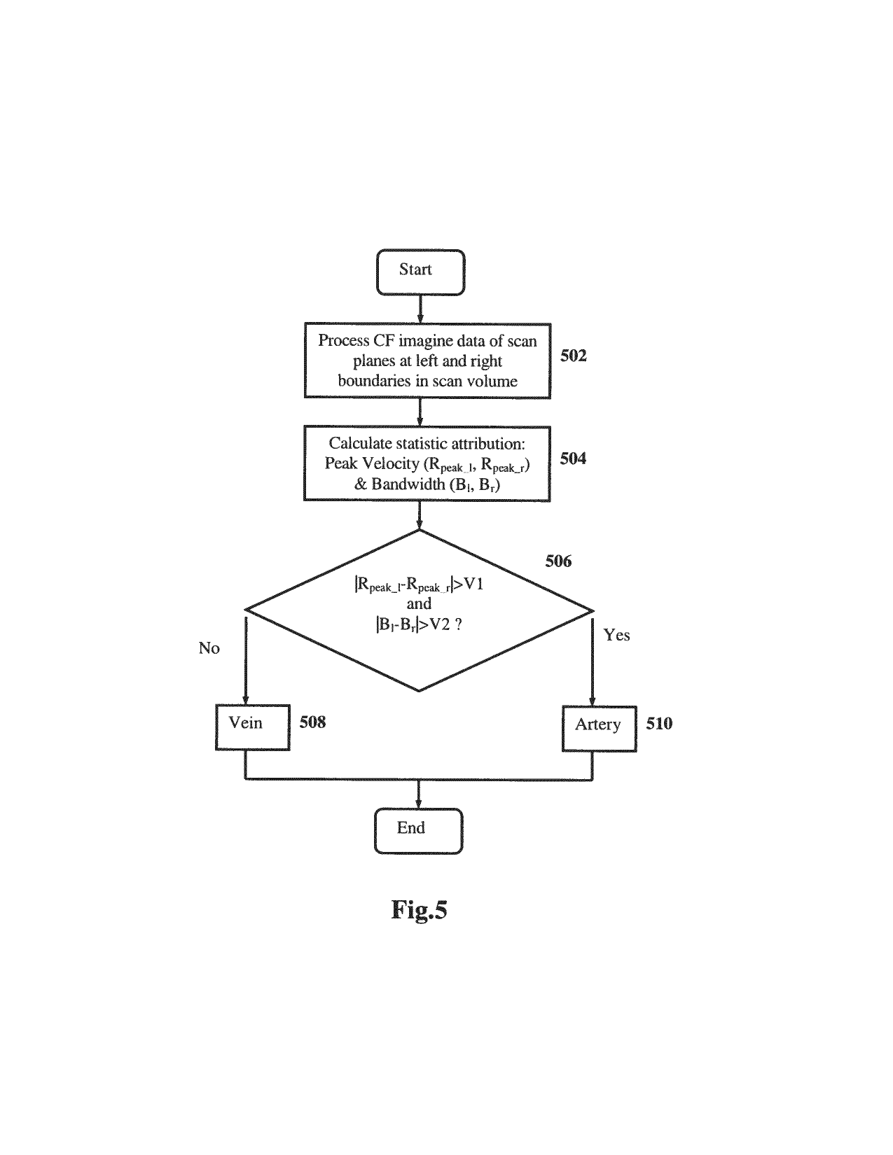

FIG. 5 illustrates a method for determining the type of the blood vessel according to an embodiment of the present invention. According to embodiments of the present invention, the blood vessel characteristics such as a vein or artery are calculated by calculating time stable or pulsatile attributes. If it is a pulsatile flow, then it is an artery; otherwise a vein. Then corresponding color dot is assigned to the blood vessel, blue for vein and red for artery.

At Step 502, the CF image data of the CF mode scan planes is processed. According to an embodiment of the present invention, the CF image data of the CF mode scan planes are combined to form ultrasound image data; the ultrasound image data is demodulated to yield the demodulated ultrasound image data; and the demodulated ultrasound image data is spectrum analyzed to yield the velocity V_{i,j} of the blood flow, where (i, j) ∈ the blood vessel region. Next, the peak velocity Rₚₑₐₖ and the bandwidth B of the blood flow are calculated.

Rₚₑₐₖ=max (|V_{i,j}|), where (i, j)∈ the blood vessel region.

B=Stdev (|V_{i,j}|), where (i, j) ∈ the blood vessel region.

|V_{i,j}| refers to the absolute value of V_{i,j}, the function max ( ) means to take the maximum value of the parameter in parentheses, and the function Stdev ( ) means to calculate the variance of the parameter in parentheses.

Processing the CF image data of the CF mode scan planes at the left boundary and the right boundary shown in FIG. 2 respectively as described above yields the peak velocities Rₚₑₐₖ_{_{—}}₁ and Rₚₑₐₖ_{_{—}}ᵣ of the blood flow at the left boundary and the right boundary and the bandwidths B₁ and Bᵣ of the blood flow at the left boundary and the right boundary.

At Step 504, it is determined whether the absolute value (Rₚₑₐₖ_{_{—}}₁-Rₚₑₐₖ_{_{—}}ᵣ|) of the difference between the peak velocity Rₚₑₐₖ_{_{—}}₁ of the blood flow at the left boundary and the peak velocity R_{peak r} of the blood flow at the right boundary is larger than the first predetermined threshold V1, and whether the absolute value (|B₁−Bᵣ|) of the difference between the bandwidth B₁ of the blood flow at the left boundary and the bandwidth Bᵣ of the blood flow at the right boundary is larger than the second predetermined threshold V2. If the result of the above determination is positive, it is determined that the blood vessel is a vein at Step 510. Otherwise, it is determined that the blood vessel is an artery at Step 508.

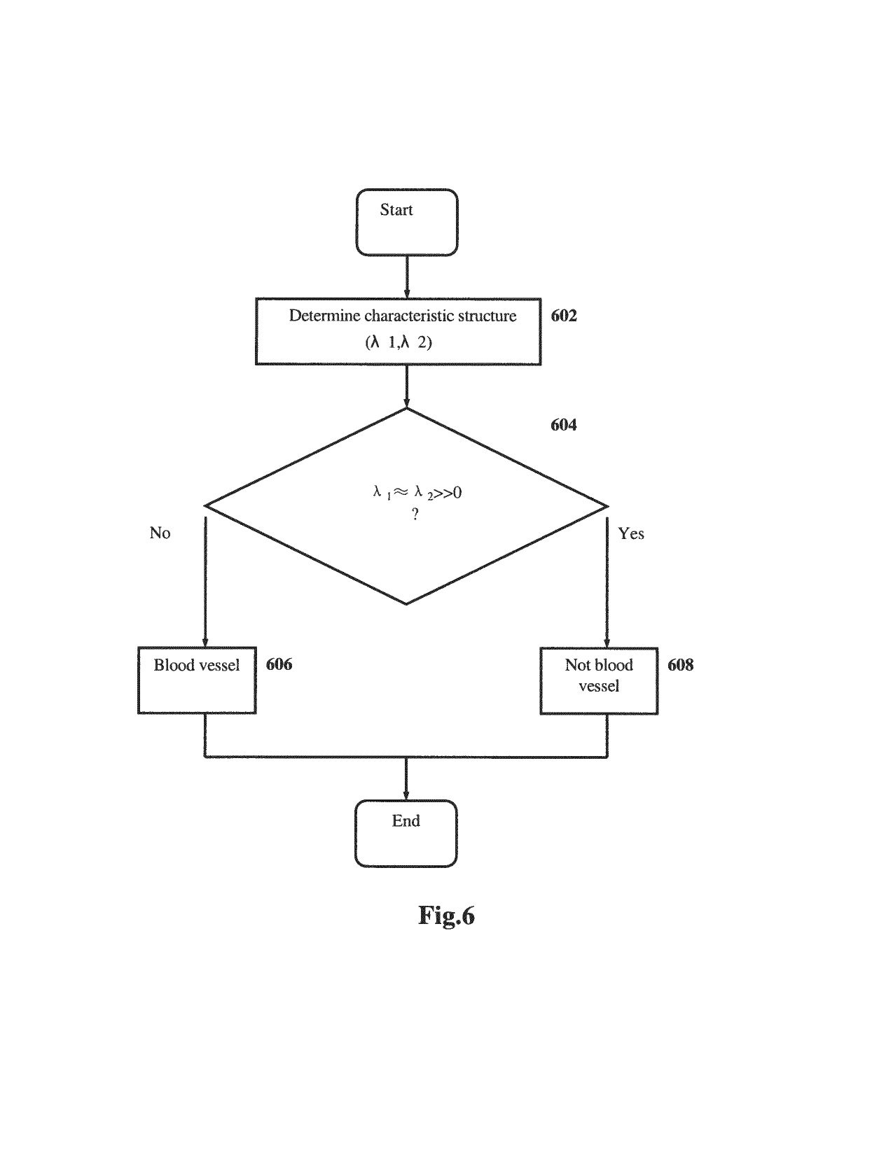

FIG. 6 illustrates a method for determining the position of the blood vessel according to an embodiment of the present invention. In an embodiment of the present invention, the position of the blood vessel in the scan space is determined based on the B mode volume image data. At Step 602, the characteristic structure of the blood vessel cross-section image in the scan plane image data of the B mode volume image data is determined. In an embodiment, boundary scan plane image data of two boundary scan planes of the scan volume is used to determine the characteristic structure of the blood vessel cross-section image. Further, in an embodiment, the circular dark region, which corresponds to the cross-section of the blood vessel, in the boundary scan image data of two boundary scan planes of the scan volume is determined.

Optionally, the characteristic structure of the blood vessel cross-section image is determined based on a Hessian matrix. The Hessian matrix is as follows:

$$H ( f ) = [ \begin{matrix}\frac{{\partial }^{2} f}{\partial {x}_{1}^{2}} & \frac{{\partial }^{2} f}{\partial {x}_{1} \partial {x}_{2}} & … & \frac{{\partial }^{2} f}{\partial {x}_{1} \partial {x}_{n}} \\ \frac{{\partial }^{2} f}{\partial {x}_{2} \partial {x}_{1}} & \frac{{\partial }^{2} f}{\partial {x}_{2}^{2}} & … & \frac{{\partial }^{2} f}{\partial {x}_{2} \partial {x}_{n}} \\ ⋮ & ⋮ & ⋱ & ⋮ \\ \frac{{\partial }^{2} f}{\partial {x}_{n} \partial {x}_{1}} & \frac{{\partial }^{2} f}{\partial {x}_{n} \partial {x}_{2}} & … & \frac{{\partial }^{2} f}{\partial {x}_{n} \partial {x}_{n}}\end{matrix} ]$$

where, n is a dimension, and x, is a vector along the iₜₕ dimension.

According to an embodiment of the present invention, a two-dimensional Hessian matrix is used to determine the characteristic structure f the blood vessel cross-section image, and whereby the Hessian matrix above is simplified as:

$${H}_{2} ( f ) = [ \begin{matrix}\frac{{\partial }^{2} f}{\partial {x}^{2}} & \frac{{\partial }^{2} f}{\partial x \partial y} \\ \frac{{\partial }^{2} f}{\partial y \partial x} & \frac{{\partial }^{2} f}{\partial {y}^{2}}\end{matrix} ]$$

where, x is an ultrasound sample direction, and y is an ultrasound beam direction.

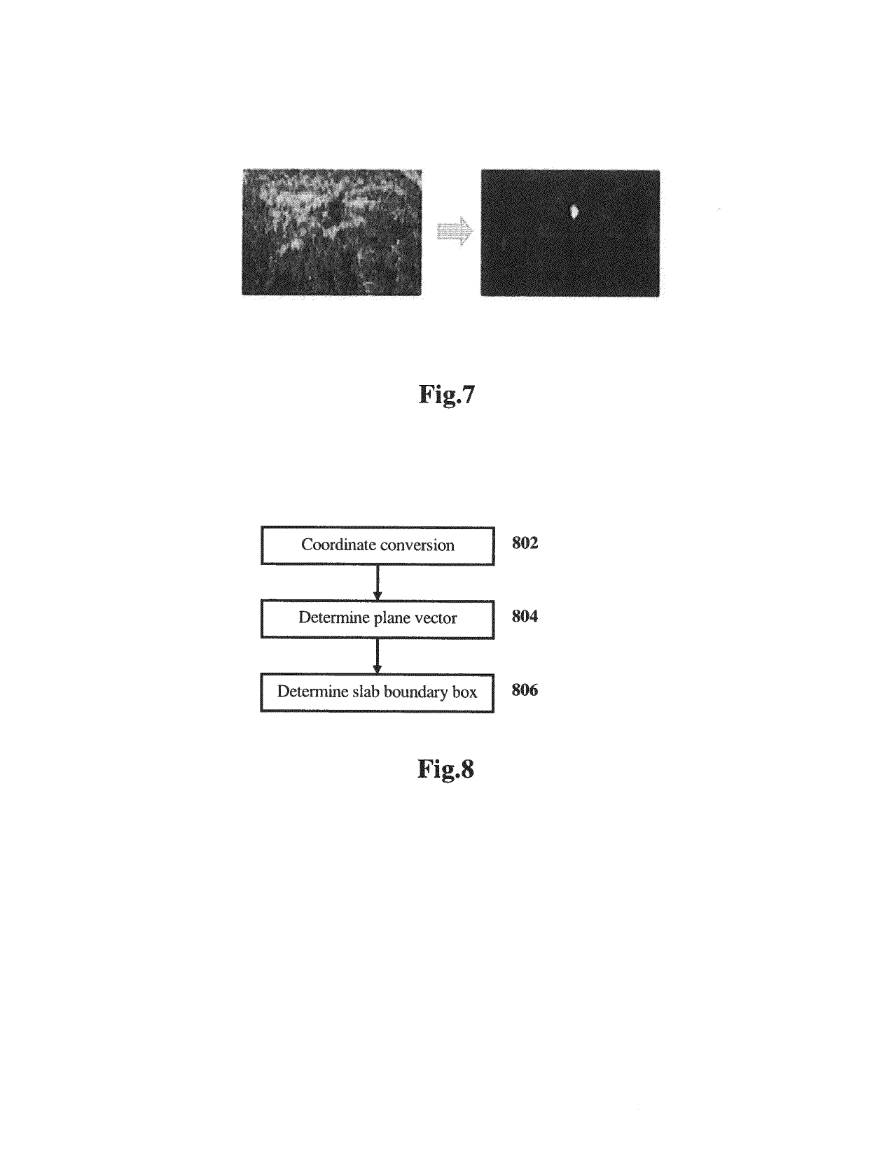

FIG. 7 illustrates comparison between the ultrasound image based on Hessian matrix processing and the ultrasound image before the Hessian matrix processing according to an embodiment of the present invention, in which the view on the left refers to the ultrasound image before the Hessian matrix processing and the view on the right refers to the ultrasound image after the Hessian matrix processing. In the image processed by the Hessian matrix, the highlighted white region refers to the blood vessel region.

At Step 604, it is determined whether the region that satisfies the characteristic structure of the blood vessel exists in the scan plane image data of the B mode volume image data. If the region that satisfies the characteristic structure of the blood vessel exists in the scan plane image data, the blood vessel region is determined, and thereby the position of the blood vessel is determined. According to an embodiment of the present invention, it is determined whether the eigenvalues λ₁ and λ₂ of the Hessian matrix in the ultrasound sample direction x and the ultrasound beam direction y satisfy the condition λ₁≈λ₂>>0; and if λ₁ and λ₂ satisfy the above condition, the blood vessel region is determined, and thereby the position of the blood vessel is determined.

The position of the blood vessel may be represented by the center point position of the blood vessel and the radius/diameter/thickness of the blood vessel. According to an embodiment of the present invention, the center point of the blood vessel is calculated based on the determined blood vessel region. For example, the centroid of the blood vessel region is calculated, and the thus calculated centroid is used as the center point of the blood vessel.

According to an embodiment of the present invention, two CF mode scan planes may be set up at the boundary of the scan volume range in the volume ultrasound scan mode to acquire the image data of two CF mode planes. The tissue image data in the B mode volume image data is excluded based on the image data of two CF mode planes. According to an embodiment of the present invention, the CF mode image data may also be used to determine the position of the blood vessel.

FIG. 8 illustrates a method for defining a slab boundary box according to an embodiment of the present invention. According to this embodiment of the present invention, the center position of the blood vessel is converted from the beam space to the Cartesian space, to determine the plane vector for generating the slab boundary box in the Cartesian space, and further generate the slab boundary box of the blood vessel.

At Step 802, the obtained blood vessel center point coordinates (n1, n2, n3) are converted from the beam space to the Cartesian space. The beam space corresponds to a three-dimension matrix in which the size of the three-dimension matrix is [N1×N2×N3], where N1 is the number of sampling points (points) on each beam, N2 is the number of electronic beams (lines), and N3 is the number of frames (plane) that are swept as the 4D probe swings (frame size=N1×N2). In the beam space, the center point of the blood vessel is represented by coordinates (n1, n2, n3), where the value of n1 ranges from 1 to N1, the value of n2 ranges from 1 to N2, and the value of n3 ranges from 1 to N3.

According to an embodiment of the present invention, the conversion of the obtained blood vessel center coordinates (n1, n2, n3) from the beam space to the Cartesian space includes a 3D conversion from the beam apace to the acquisition coordinate system (or sweeping coordinate system) and a 3D conversion from the acquisition coordinate system to the Cartesian coordinate system.

(1) Conversion from Beam Space to Acquisition Coordinate System.

In embodiments of the present invention, the acquisition coordinates correspond to the cylindrical coordinates. For the integer values of the beam space coordinates (n1, n2, n3) corresponding to the voxel position in the beam space, the cylindrical coordinates in the cylindrical acquisition coordinate system are given by:

r=n₁ for n₁={1, 2, . . . , N₁}

s=shotangle(n₂) for n₂={1, 2, . . . , N₂}

β=BImageAngles(n₃) for n₃={1, 2, . . . , N₃}

where, r, s and β refer to a radial distance in the cylindrical coordinates (in the beam sample direction), a distance in the beam array direction and a corresponding scan elevation respectively. shotangles is a one-dimension vector that is incremented on the basis of the voxel size in accordance with the offset of each scan line position (angle); and BimageAngles is a one-dimension vector that is incremented in accordance with the probe scan elevation in the unit of arc.

According to an embodiment of the present invention, shotangles(n)={0.0, 0.1, 0.2 . . . }, and BimageAngles (n)={0.0, 0.02, 0.04, 0.06 . . . } Shotangles and BimageAngles may also employ vectors with other values, and may not necessarily be subjected to sampling with the equal sample interval.

(2) Conversion from Acquisition Coordinate System to Cartesian Coordinate System.

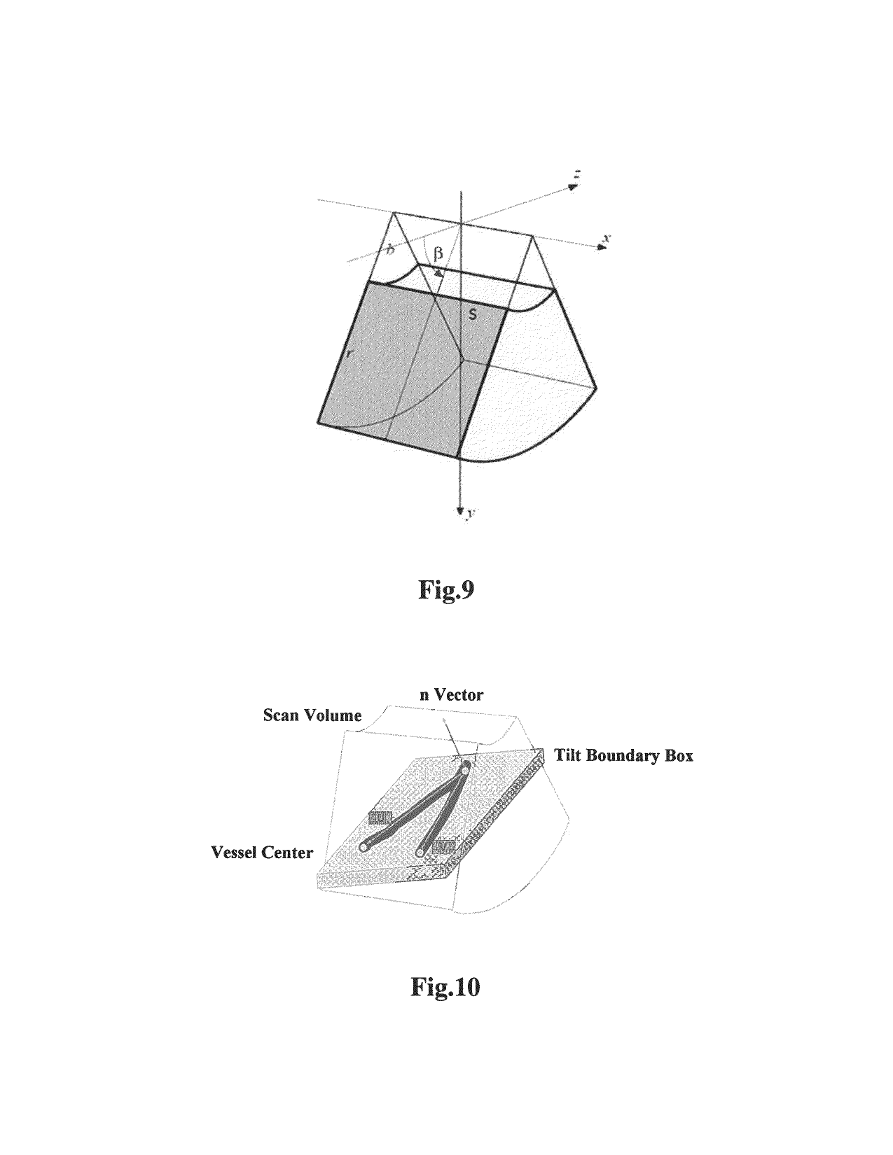

FIG. 9 illustrates conversion from the acquisition coordinate system to the Cartesian coordinate system according to an embodiment of the present invention. The equation for the conversion from the cylindrical coordinates of the cylindrical acquisition coordinate system to the coordinates of the Cartesian coordinate system is:

x=δs

y=δ(b+r)sin(β)

z=δ(b+r)cos(β)

where, r, s and β refer to the radial distance in the cylindrical coordinates (in the beam sample direction), a distance in the beam array direction and corresponding scan elevation coordinates, respectively, x, y and z correspond to coordinates of the Cartesian coordinate system, δ is a scaling factor of the three-dimension data field, and b is an offset from the probe surface to the scan starting position. According to an embodiment of the present invention, the value range of δ>=1.0. b may be defined by a user. According to an embodiment of the present invention, b may take a value in the range from 0.0 to 7.0 cm. It shall be pointed out that δ and b are not limited to the above-mentioned values, and may also take other values.

Optionally, after the coordinate conversions in the above steps (1) and (2) are finished, a 3D rotating conversion may also be performed as follows.

$$( \begin{matrix}{x}^{′} \\ {y}^{′} \\ {z}^{′}\end{matrix} ) = [ \begin{matrix}1 & 0 & 0 \\ 0 & cos α & sin α \\ 0 & - sin α & cos α\end{matrix} ] [ \begin{matrix}cos β & 0 & - sin β \\ 0 & 1 & 0 \\ sin β & 0 & cos β\end{matrix} ] [ \begin{matrix}cos γ & sin γ & 0 \\ - sin γ & cos γ & 0 \\ 0 & 0 & 1\end{matrix} ] ( \begin{matrix}x \\ y \\ z\end{matrix} )$$

α, β and γ are rotating angles around X, Y and Z axes, and take values in the range from 0 to 2π. Values of α, β and γ depend on the perspective of interaction selected by the user. (x′, y′, z′) are the coordinates of the final blood vessel center point in the Cartesian coordinate system.

At Step 804, after the conversion of the blood vessel center point from the beam space coordinates (n1, n2, n3) into the Cartesian coordinate system coordinates (x, y, z) (or (x′, y′, z′)) is finished, the plane vector of the surface of the slab boundary box is determined. According to an embodiment of the present invention, the plane vector of the pair of outer surfaces of the slab boundary box may be determined based on the direction of the center point connecting line of the blood vessel.

FIG. 10 illustrates a schematic diagram of the determination of the plane vector of the pair of outer surfaces of the slab boundary box based on the direction of the center point connecting line of the blood vessel according to an embodiment of the present invention. As shown in FIG. 10, the plane vector n is calculated from two blood vessel center line vectors u and v. (x1, y1, z1), (x2, y2, z2), (x3, y3, z3) are coordinates of three blood vessel center points in the Cartesian coordinate system.

$$\begin{matrix}u = ( {u}_{x} , {u}_{y} , {u}_{z} ) \\ = ( x 2 - x 1 , y 2 - y 1 , z 2 - z 1 )\end{matrix}$$

$$\begin{matrix}v = ( {v}_{x} , {v}_{y} , {v}_{z} ) \\ = ( x 3 - x 1 , y 3 - y 1 , z 3 - z 1 )\end{matrix}$$

$$\begin{matrix}n = u \times v \\ = \vert \begin{matrix}i & j & k \\ {u}_{x} & {u}_{y} & {u}_{z} \\ {v}_{x} & {v}_{y} & {v}_{z}\end{matrix} \vert\end{matrix}$$

Optionally, for the blood vessel having branches, the two blood vessel center line vectors u and v may be vectors in the directions of two lines that connect the blood vessel center point on the blood vessel crossing section and each of the blood vessel center points on two branch sections respectively. In an embodiment, the blood vessel center point on the blood vessel crossing section and the blood vessel center points on two branch sections are taken from the blood vessel center points of two boundary scan planes of the scan volume.

Optionally, for the curvous blood vessels, the two blood vessel center line vectors u and v may be vectors in the directions of two lines that connect each of the blood vessel center points near two blood vessel center points of the blood vessel at two ends of the scan volume and one blood center point on the blood vessel middle section in the scan volume respectively. In an embodiment, the blood vessel center point on the blood vessel middle section is the blood vessel center farthest from the straight line connecting the blood vessel center points of two boundary scan planes of the scan volume.

Optionally, for relatively straight blood vessels, one of the two blood vessel center line vectors u and v may be a vector in the direction of the line that connects any two blood vessel center points of the blood vessel in the scan volume, and the other may be a predetermined vector which can be set up in accordance with the user's preference. For example, in an embodiment, the predetermined vector may be a vector oriented in the direction vertical to the screen, thus the observation range may be larger or be set up in a different manner.

At Step 806, the slab boundary box is determined such that the plane vector of the pair of outer surfaces of the slab boundary box is the above-mentioned plane vector n. The pair of outer surfaces of the slab boundary box extend outwardly from the blood vessel center point in the direction of the plane vector n and the direction opposite to the plane vector n, respectively, such that the slab boundary box covers the whole blood vessel. In an embodiment, the pair of outer surfaces of the slab boundary box extend outwardly from the blood vessel center point in the direction of the plane vector n and the direction opposite to the plane vector n, respectively, such that the slab boundary box just covers the whole blood vessel. According to an embodiment of the present invention, lengths extending in two directions are respectively equal to the radius of the blood vessel, and the value of the radius is obtained from detection of the blood vessel in the CF and B modes, and also needs to undergo the above-mentioned two-step coordinate conversion. According to an embodiment of the present invention, lengths extending in two directions may be larger than the radius of the blood vessel. For example, it may be slightly larger than the radius of the blood vessel, or may be 1.1-2.0 times as long as the radius of the blood vessel, or even more. According to an embodiment of the present invention, the intersection of the slab boundary box and the scan volume is used as VOI (volume of interest) which is to be rendered.

In the ultrasound volume image, a blood vessel appears as a dark image, and the tissues around the blood vessel also appear as dark images, thereby interfering with the presentation of the blood vessel image. According to the embodiments of the present invention, 3D rendering VOI is defined based on the position and size of the blood vessel. Thus, some dark tissues are excluded from the rendered VOI, while the valid blood vessel information of the corresponding blood vessel is included in the rendered VOI, and only the blood vessel images in the rendered VOI are processed and displayed.

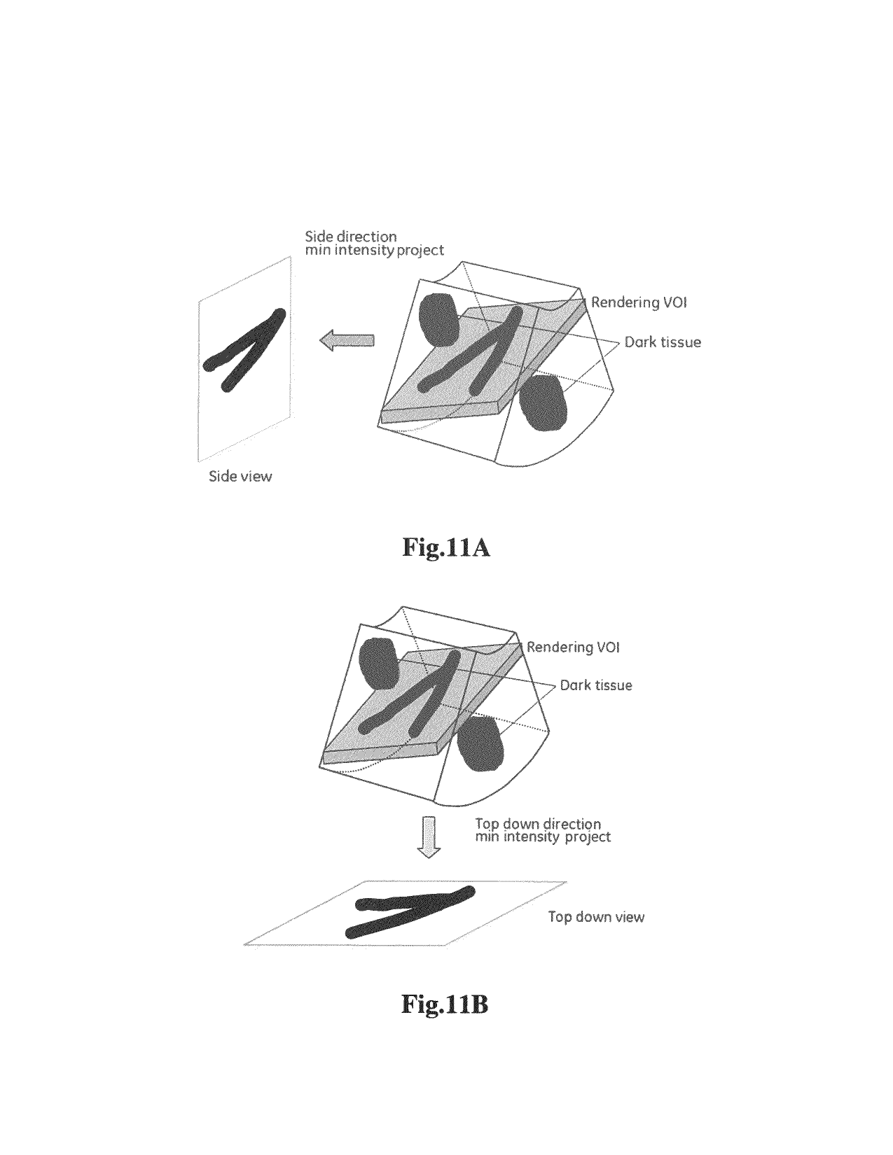

FIGS. 11A and 11B illustrate respectively the minimum intensity projection images of a side view and a top view according to an embodiment of the present invention, in which FIG. 11A is a side view of the blood vessel and FIG. 11B is a top view of the blood vessel. The top view shows the blood vessel offset position from a top-down direction, and the side view shows the blood vessel depth position from a left-to-right direction. As seen from FIG. 11A and FIG. 11B, since the embodiments of the present invention are to render the region in the slab boundary box, the dark tissues absent from the slab boundary box do not appear in the rendered image.

The method according to embodiments of the present invention may be embodied by means of software, hardware and firmware or a combination of software, hardware and firmware, and the software, hardware and firmware for embodying the method may be distributed in different units or integrated in one unit.

The method according to embodiments of the present invention may be accomplished automatically.

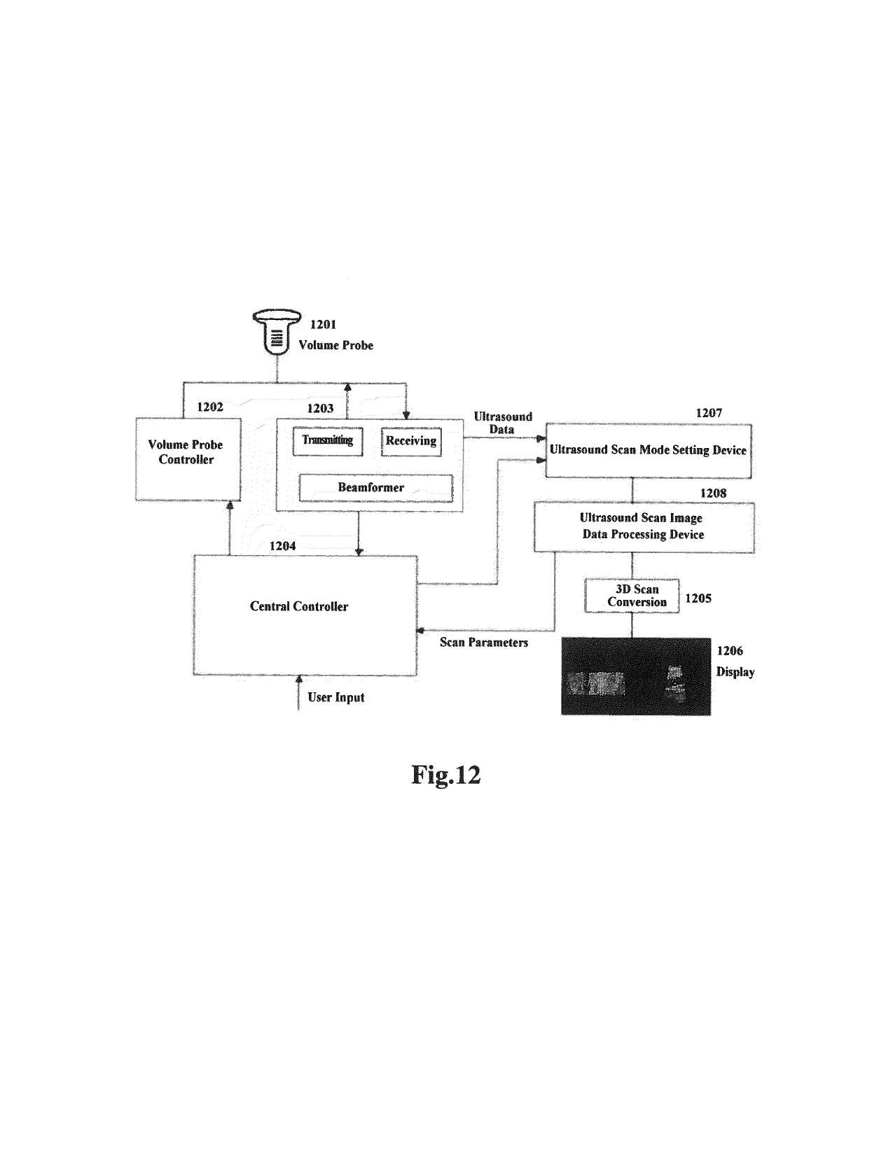

FIG. 12 illustrates a system for displaying ultrasound images operable to implement the present invention. The system for displaying ultrasound images comprises a volume probe for transmitting and receiving an ultrasound signal; a volume probe controller for controlling operation of the volume probe; a beamformer for forming an ultrasound beam; an ultrasound scan mode setting device for setting different ultrasound scan modes; an ultrasound scan image data processing device for generating rendered VOI of an ultrasound scanned object; a display for displaying the rendered VOI from the ultrasound scan data processing device; a central processing unit for processing data from the beamformer and the ultrasound scan processing device and controlling the volume probe controller and the ultrasound scan mode setting device.

According to an embodiment of the present invention, the central controller receives scan parameters from the ultrasound scan data processing device, and controls the operation of the volume probe through the volume probe controller.

According to an embodiment of the present invention, the ultrasound scan mode setting device further comprises a B mode setting unit for sampling an object to be imaged by setting the B mode, so as to obtain the B mode volume image data in the B mode; and a CF mode setting unit for sampling an object to be imaged by setting the CF mode, so as to obtain the CF mode image data in the CF mode. Optionally, the ultrasound scan mode setting device further comprises a memory for storing the B mode volume image data in the B mode and/or the CF mode image data in the CF mode. Optionally, the CF mode image data may be the CF mode image data of two boundary scan planes of the scan volume. Alternatively, a memory may be provided outside of the ultrasound scan mode setting device, and the ultrasound scan mode setting device may externally store the B mode volume image data in the B mode and/or the CF mode image data in the CF mode.

According to an embodiment of the present invention, the ultrasound scan data processing device further comprises an object locating unit for locating the position of the blood vessel based on the ultrasound volume image data; a slab boundary box determination unit for defining the slab boundary box surrounding the blood vessel based on the position of the blood vessel; and a rendering unit for rendering the region surrounded by the slab boundary box to be displayed on the display.

According to an embodiment of the present invention, the object locating unit comprises an object determination unit for determining whether the region that satisfies the predetermined structure feature exists in the scan plane image data of the ultrasound volume image data, and if yes, determining that the region belongs to the blood vessel. According to the embodiments of the present invention, the predetermined characteristic structure is a dark circle. Determining whether the structure feature exists in the scan plane image data of the ultrasound volume image data includes, based on the second order Hessian matrix

$${H}_{2} ( f ) = [ \begin{matrix}\frac{{\partial }^{2} f}{\partial {x}^{2}} & \frac{{\partial }^{2} f}{\partial x \partial y} \\ \frac{{\partial }^{2} f}{\partial y \partial x} & \frac{{\partial }^{2} f}{\partial {y}^{2}}\end{matrix} ]$$

determining whether a region in the scan plane image data of the ultrasound volume image data satisfies the above-mentioned predetermined structure feature. If the eigenvalues λ₁ and λ₂ in the ultrasound sample direction x and the ultrasound beam direction y are approximately equal to each other and much larger than 0, it is determined that the region belongs to the blood vessel.

According to an embodiment of the present invention, the ultrasound scan image data processing device further comprises a center point determination unit for determining the center point of the blood vessel region as the center point of the blood vessel. The ultrasound scan image data processing device may further comprise a coordinate conversion unit for converting the center point of the blood vessel into the Cartesian coordinate system. The slab boundary box determination unit may further comprise a plane vector determination unit for determining the plane vector n of the pair of outer surfaces of the slab boundary box based on the direction of the center point connecting line of the blood vessel; and a slab boundary box setting unit for extending the pair of outer surfaces of the slab boundary box outwardly from the center point of the blood vessel in the direction if the plane vector n and the direction opposite to the plane vector n, respectively, so as to surround the whole blood vessel in the scan volume.

According to an embodiment of the present invention, the plane vector determination unit determines the plane vector n as follows:

n=u×v

where, u and v directions are parallel to directions of two lines each connecting two center points of the blood vessel in the scan volume respectively; and if the blood vessel looks straight, the predetermined vector direction is used instead of the vector v direction. Two center points of one of the lines and two center points of the other line are selected from the group consisting of the center point of the blood vessel on the scan plane in the fork of the blood vessel in the scan volume, the center points of the blood vessel on two boundary scan planes in the scan volume, and the center point of the blood vessel on a scan plane in the middle of the blood vessel in the scan volume.

The ultrasound scan image data processing device may further comprise an object type determination unit for determining the type of the blood vessel based on the CF mode image data.

Optionally, various components of the system for displaying ultrasound images according to the embodiments of the present invention are distributed in the central processing unit or partly distributed in the central processing unit. Furthermore, various components of the system for displaying ultrasound images according to the embodiments of the present invention may also be distributed in other devices, such as the volume probe controller, the beamformer, of the system, or partly distributed in these devices.

According to embodiments of the present invention, the various devices, units and components described above may be combined in various manners, and not limited to the above-mentioned manners, wherein some of the combinations may function as relatively independent devices. For example, an embodiment of the present invention is to provide an ultrasound volume image data processing device comprising an ultrasound volume image data acquisition unit for acquiring the ultrasound volume image data in the scan volume, wherein the blood vessel is located in the scan volume; an object locating unit for locating the position of the blood vessel based on the ultrasound volume image data; a slab boundary box determination unit for defining the slab boundary box surrounding the blood vessel based on the position of the blood vessel; and a rendering unit for rendering the region surrounded by the slab boundary box.

In an embodiment, the ultrasound volume image data acquisition unit comprises a B mode volume image data acquisition unit for acquiring the B mode volume image data in the B mode; and a CF mode image data acquisition unit for acquiring the CF mode image data in the CF mode.

In an embodiment, the CF mode image data is the CF mode image data of two boundary scan planes of the scan volume.

In an embodiment, the object locating unit comprises an object determination unit for determining whether the region that satisfies the predetermined structure feature exists in the scan plane image data of the ultrasound volume image data, and if yes, determining that the region belongs to the blood vessel.

In an embodiment, the predetermined characteristic structure is a dark circle.

In an embodiment, the scan plane image data in the ultrasound volume image data is the boundary scan plane image data of two boundary scan planes of the scan volume.

In an embodiment, determining whether the structure feature exists in the scan plane image data of the ultrasound volume image data includes determining whether the region in the scan plane image data of the ultrasound volume image data satisfies the predetermined structure feature based on the second order Hessian matrix, and determining that the region belongs to the blood vessel if the eigenvalues λ₁ and λ₂ in the ultrasound sample direction x and the ultrasound beam direction y are approximately equal to each other and much larger than 0.

In an embodiment, a center point determination unit is further comprised for determining the center point of the region of the blood vessel as the center point of the blood vessel.

In an embodiment, a coordinate conversion unit is further comprised for converting the center point of the blood vessel into the Cartesian coordinate system.

In an embodiment, the slab boundary box determination unit comprises a plane vector determination unit for determining the plane vector n of the pair of outer surfaces of the slab boundary box based on the direction of the center point connecting line of the blood vessel; and a slab boundary box setting unit for extending the pair of outer surfaces of the slab boundary box outwardly from the center point of the blood vessel in the direction of the plane vector n and the direction opposite to the plane vector n, so as to surround the whole blood vessel in the scan volume.

In an embodiment, the plane vector determination unit determines the plane vector n in accordance with n=u×v, where, the directions of vectors u and v are parallel to directions of two lines each connecting two center points of the blood vessel in the scan volume, respectively; and if the blood vessel looks straight, the predetermined vector direction is used instead of the v direction.

In an embodiment, two center points of one of the lines and two center points of the other line are selected from the group consisting of the center point of the blood vessel on the scan plane in the fork of the blood vessel in the scan volume, the center points of the blood vessel on two boundary scan planes in the scan volume, and the center point of the blood vessel on the scan plane in the middle of the blood vessel in the scan volume.

In an embodiment, an object type determination unit is further comprised for determining the type of the blood vessel based on the CF mode image data.

Embodiments of the present invention are to address deficiencies of the prior art by providing a method and a device for ultrasound volume image data processing. The method and the device for ultrasound volume image data processing according to embodiments of the present invention comprise acquiring ultrasound volume image data in the scan volume in which a line-like object is located; locating a position of the line-like object based on the ultrasound volume image data; defining a slab boundary box surrounding the line-like object based on the position of the line-like object; and rendering a region surrounded by the slab boundary box.

In an embodiment, acquiring the ultrasound volume image data includes acquiring B mode volume image data in a B mode and CF mode image data in a CF mode.

In an embodiment, the CF mode image data is the CF mode image data of two boundary scan planes of the scan volume.

In an embodiment, locating the position of the line-like object includes determining whether a region that satisfies a predetermined structure feature exists in the scan plane image data of the ultrasound volume image data; and determining that the region belongs to the line-like object if it satisfies the predetermined structure feature.

In an embodiment, the predetermined characteristic structure is a dark circle.

In an embodiment, the scan plane image data in the ultrasound volume image data is boundary scan plane image data of two boundary scan planes of the scan volume.

Determining whether the structure feature exists in the scan plane image data of the ultrasound volume image data includes based on a second order Hessian matrix

$${H}_{2} ( f ) = [ \begin{matrix}\frac{{\partial }^{2} f}{\partial {x}^{2}} & \frac{{\partial }^{2} f}{\partial x \partial y} \\ \frac{{\partial }^{2} f}{\partial y \partial x} & \frac{{\partial }^{2} f}{\partial {y}^{2}}\end{matrix} ]$$

determining whether a region in the scan plane image data of the ultrasound volume image data satisfies the predetermined structure feature; and determining that the region belongs to the line-like object if eigenvalues λ₁ and λ₂ in an ultrasound sample direction x and an ultrasound beam direction y are approximately equal to each other and much larger than 0.

Further, the center point of the region of the line-like object is determined as a center point of the line-like object.

Furthermore, the center point of the line-like object is converted into the Cartesian coordinate system.

In an embodiment, defining the slab boundary box surrounding the line-like object includes determining a plane vector n of a pair of outer surfaces of the slab boundary box based on a direction of a center point connecting line of the line-like object (i.e. a line passing through center points of cross-sections of the line-like object); and extending the pair of outer surfaces of the slab boundary box outwardly from the center point of the line-like object in a direction of the plane vector n and a direction opposite to the plane vector n respectively, so as to surround the whole line-like object in the scan volume.

In an embodiment, the plane vector n is determined as follows:

n=u×v

where vector u and v directions are respectively parallel to directions of two straight lines, each of which connects two center points of the line-like object, in the scan volume, and if the line-like object looks straight, a predetermined vector direction is used instead of the v direction.

In an embodiment, two center points of one of the straight lines and two center points of the other of the straight lines are selected from the group consisting of: a center point of the line-like object on a scan plane in the fork of the line-like object in the scan volume, center points of the line-like object on two boundary scan planes of the scan volume, and a center point of the line-like object on a scan plane in the middle of the line-like object in the scan volume.

Furthermore, the type of the line-like object is determined based on the CF mode image data.

In an embodiment, the line-like object may be a fluid conduit.

In an embodiment, the fluid conduit may be a blood vessel.

Although the present invention has been described with reference to specific embodiments above, it is not intended that the present invention be limited to these specific embodiments. Those skilled in the art will appreciate that various modifications, equivalent substitutions and changes may be made to the present invention. For example, one step or module in the above embodiments may be implemented in two or more steps or modules; or conversely, functions of two or more steps or modules or devices in the above embodiments may be implemented in one step or module. However, these changes should fall within the scope of protection of the present invention without departing from the spirit of the present invention. In addition, some terms as used in the specification and claims of this application are to be considered as illustrative rather than restrictive in character.

What is claimed is:

1. A method for ultrasound volume image data processing, comprising:
acquiring ultrasound volume image data in a scan volume in which a line-like object is located;
locating a position of the line-like object based on the ultrasound volume image data;
defining a slab boundary box surrounding the line-like object based on the position of the line-like object; and
rendering a region surrounded by the slab boundary box.

2. The method according to claim 1, wherein acquiring the ultrasound volume image data comprises:
acquiring B mode volume image data in a B mode; and
acquiring CF mode image data in a CF mode.

3. The method according to claim 2, wherein the CF mode image data is the CF mode image data of two boundary scan planes of the scan volume.

4. The method according to claim 2, wherein locating the position of the line-like object comprises:
determining whether a region that satisfies a predetermined structure feature exists in the scan plane image data of the ultrasound volume image data; and
determining that the region belongs to the line-like object if a region that satisfies the predetermined structure feature exists in the scan plane image data of the ultrasound volume image data.

5. The method according to claim 4, wherein the predetermined structure feature is a dark circle.

6. The method according to claim 4, wherein the scan plane image data of the ultrasound volume image data is boundary scan plane image data of two boundary scan planes of the scan volume.

7. The method according to claim 4, wherein determining whether a region that satisfies a predetermined structure feature exists in the scan plane image data of the ultrasound volume image data comprises:
determining, based on a second order Hessian matrix
$${H}_{2} ( f ) = [ \begin{matrix}\frac{{\partial }^{2} f}{\partial {x}^{2}} & \frac{{\partial }^{2} f}{\partial x \partial y} \\ \frac{{\partial }^{2} f}{\partial y \partial x} & \frac{{\partial }^{2} f}{\partial {y}^{2}}\end{matrix} ] ,$$
whether a region in the scan plane image data of the ultrasound volume image data satisfies the predetermined structure feature; and
determining that the region belongs to the line-like object if eigenvalues and in an ultrasound sample direction and an ultrasound beam direction are approximately equal to each other and much larger than 0.

8. A method according to claim 7, further comprising:
determining a center point of the region of the line-like object as a center point of the line-like object.

9. The method according to claim 8, further comprising:
converting the center point of the line-like object into a Cartesian coordinate system.

10. The method according to claim 8, wherein defining the slab boundary box surrounding the line-like object comprises:
determining a plane vector of a pair of outer surfaces of the slab boundary box based on a direction of a center point connecting line of the line-like object; and
extending the pair of outer surfaces of the slab boundary box outwardly from the center point of the line-like object in a direction of the plane vector and a direction opposite to the plane vector respectively, so as to surround the whole line-like object in the scan volume.

11. The method according to claim 10, wherein the plane vector is determined as:
n=u×v,
where the direction of vector u and vector v are respectively parallel to directions of two straight lines, wherein each of the two straight lines connects two center points of the line-like object, in the scan volume; and if the line-like object is straight, a predetermined vector direction is used instead of the v direction.

12. The method according to claim 11, wherein the two center points of one of the two straight lines and the two center points of the other of the two straight lines are selected from the group comprising:
a center point of the line-like object on a scan plane in a fork of the line-like object in the scan volume;
center points of the line-like object on two boundary scan planes in the scan volume; and
a center point of the line-like object on a scan plane in a middle of the line-like object in the scan volume.

13. The method according to claim 2, further comprising:
determining a type of the line-like object based on the CF mode image data.

14. The method according to claim 2, wherein the line-like object is a fluid conduit.

15. The method according to claim 14, wherein the fluid conduit is a blood vessel.

16. An ultrasound volume image data processing device, the device comprising:
an ultrasound volume image data acquisition unit configured to acquire ultrasound volume image data in a scan volume in which a line-like object is located;
an object locating unit configured to locate a position of the line-like object based on the ultrasound volume image data;
a slab boundary box determination unit configured to define a slab boundary box surrounding the line-like object based on the position of the line-like object; and
a rendering unit configured to render a region surrounded by the slab boundary box.

17. The device according to claim 16, wherein the ultrasound volume image data acquisition unit comprises:
a B mode volume image data acquisition unit configured to acquire B mode volume image data in a B mode; and
a CF mode image data acquisition unit configured to acquire CF mode image data in a CF mode.

18. The device according to claim 17, wherein the CF mode image data is the CF mode image data of two boundary scan planes of the scan volume.

19. The device according to claim 17, wherein the object locating unit comprises:
an object determination unit configured to determine whether a region that satisfies a predetermined structure feature exists in scan plane image data of the ultrasound volume image data, and to determine that the region belongs to the line-like object if a region that satisfies a predetermined structure feature exists in scan plane image data of the ultrasound volume image data.

20. The device according to claim 19, wherein the predetermined structure feature is a dark circle.

21. The device according to claim 19, wherein the scan plane image data of the ultrasound volume image data is boundary scan plane image data of two boundary scan planes of the scan volume.

22. The device according to claim 19, wherein the object determination unit is further configured to, based on a second order Hessian matrix:
$${H}_{2} ( f ) = [ \begin{matrix}\frac{{\partial }^{2} f}{\partial {x}^{2}} & \frac{{\partial }^{2} f}{\partial x \partial y} \\ \frac{{\partial }^{2} f}{\partial y \partial x} & \frac{{\partial }^{2} f}{\partial {y}^{2}}\end{matrix} ] ,$$
determine whether a region in the scan plane image data of the ultrasound volume image data satisfies the predetermined structure feature, and determine that the region belongs to the line-like object if eigenvalues and in an ultrasound sample direction and an ultrasound beam direction y are approximately equal to each other and much larger than 0.

23. The device according to claim 22, further comprising:
a center determination unit configured to determine a center point of the region of the line-like object as a center point of the line-like object.

24. The device according to claim 23, further comprising:
a coordinate conversion unit configured to convert the center point of the line-like object into a Cartesian coordinate system.

25. The device according to claim 23, wherein the slab boundary box determination unit comprises:
a plane vector determination unit configured to determine a plane vector of a pair of outer surfaces of the slab boundary box based on a direction of the center point connecting line of the line-like object; and
a slab boundary box setting unit configured to extend the pair of outer surfaces of the slab boundary box outwardly from the center point of the line-like object in a direction of the plane vector and a direction opposite to the plane vector respectively, so as to surround the whole line-like object in the scan volume.

26. The device according to claim 25, wherein the plane vector determination unit is configured to determine the plane vector as follows:
n=u×v
where the direction of vector u and vector v are respectively parallel to directions of two straight lines, wherein each of the two straight lines connects two center points of the line-like object, in the scan volume; and if the line-like object is straight, a predetermined vector direction is used instead of the v direction.

27. The device according to claim 26, wherein the two center points of one of the two straight lines and the two center points of the other of the two straight lines are selected from the group consisting of:
a center point of the line-like object on a scan plane in a fork of the line-like object in the scan volume;
center points of the line-like object on two boundary scan planes in the scan volume; and
a center point of the line-like object on a scan plane in a middle of the line-like object in the scan volume.

28. The device according to claim 17, further comprising:
an object type determination unit configured to determine a type of the line-like object based on the CF mode image data.

29. The device according to claim 17, wherein the line-like object is a fluid conduit.

30. The device according to claim 29, wherein the fluid conduit is a blood vessel.
